(19) United States
(12) Patent Application Publication
(10) Pub. No.: US 2024/0386701 A1
(43) Pub. Date: Nov. 21, 2024

(54) MEDICAL IMAGE PROCESSING APPARATUS AND ENDOSCOPE SYSTEM

(71) Applicant: FUJIFILM Corporation, Tokyo (JP)

(72) Inventor: Shumpei KAMON, Kanagawa (JP)

(73) Assignee: FUJIFILM Corporation, Tokyo (JP)

(21) Appl. No.: 18/664,264

(22) Filed: May 14, 2024

(30) Foreign Application Priority Data
May 16, 2023 (JP) 2023-080690

(51) Int. Cl. G06V 10/764; A61B 1/00; G06V 10/25; G06V 10/70; G06V 10/94; G16H 50/20

(52) U.S. Cl. CPC G06V 10/764; A61B 1/000094; G06V 10/25; G06V 10/87; G06V 10/955; G16H 50/20; G06V 2201/03

(57) ABSTRACT

The medical image processing apparatus includes: a processor; a classifier that classifies an imaged part in a medical image; and a plurality of recognizers that recognize a region of interest in the medical image and correspond to each imaged part, in which the processor is configured to: acquire a plurality of medical images having a first medical image and a second medical image acquired at a later time point than the first medical image; acquire a classification result of the imaged part in the first medical image from the classifier; select the recognizer corresponding to the classification result of the first medical image from the plurality of recognizers; and acquire a recognition result of the region of interest in the second medical image from the selected recognizer.

### CROSS-REFERENCE TO RELATED APPLICATIONS

This application claims priority under 35 U.S.C § 119 (a) to Japanese Patent Application No. 2023-080690 filed on 16 May 2023. The above application is hereby expressly incorporated by reference, in its entirety, into the present application.

### BACKGROUND OF THE INVENTION

### 1. Field of the Invention

The present invention relates to a medical image processing apparatus and an endoscope system.

### 2. Description of the Related Art

Endoscopes and ultrasound diagnostic apparatuses observe a plurality of parts in a single examination. In a case of implementing diagnostic support functions using machine learning such as lesion detection and lesion discrimination for such medical apparatuses, there is a problem in that the same machine learning model cannot achieve a desired performance because image features differ depending on the part. In such a case, a configuration can be considered in which a trained model optimized for each part is installed and the trained model is appropriately used to support the examination depending on a classification result of the part being examined.

Since a user during the examination is required to perform complicated procedures, it is preferable to have a configuration in which selection and switching are performed automatically according to the classification results. That is, apart from an examination support model for each part, a trained model for part classification is prepared to classify imaged parts from medical images, and a trained model for examination support is selected according to the result. Specifically, in JP2013-123528A, lesion position information is acquired from a computed tomography (CT) image or a magnetic resonance imaging (MRI) image, and identity of the lesion is determined according to the position. In JP2012-249956A, a part is specified from image data of a capsule endoscope, and analysis is performed according to the part.

### SUMMARY OF THE INVENTION

On the other hand, JP2013-123528A targets still images and does not assume processing for moving images, and in JP2012-249956A, high-speed processing is difficult because part specification and analysis are performed on the same image, but in a case of providing support using machine learning or the like to a modality that performs a dynamic examination such as an endoscope or an ultrasound diagnostic apparatus, it is required to output processing results in real time during the examination.

An object of the present invention is to provide a medical image processing apparatus and an endoscope system that can achieve both selecting an optimal examination support model for each part of a medical image and performing real-time processing.

According to an aspect of the present invention, there is provided a medical image processing apparatus comprising: a processor; a classifier that classifies an imaged part in a medical image; and a plurality of recognizers that recognize a region of interest in the medical image and correspond to each imaged part, in which the processor is configured to: acquire a plurality of medical images having a first medical image and a second medical image acquired at a later time point than the first medical image; acquire a classification result of the imaged part in the first medical image from the classifier; select the recognizer corresponding to the classification result of the first medical image from the plurality of recognizers; and acquire a recognition result of the region of interest in the second medical image from the selected recognizer.

It is preferable that classification of the imaged part in the second medical image by the classifier, and recognition of the region of interest in the second medical image by the recognizer are performed in parallel processing.

It is preferable that a notification of the recognition result is provided.

It is preferable that a display image using the recognition result is created, and the notification is provided by displaying the display image on a screen.

It is preferable that classification of the imaged part in a third medical image acquired at a later time point than the second medical image by the classifier, recognition of the region of interest in the third medical image by the recognizer based on the imaged part in the second medical image, and creation of the display image of the recognition result in the second medical image are performed in parallel processing.

It is preferable that the display image is created by superimposing the recognition result on the third medical image.

It is preferable that a notification of a type of the recognizer used to recognize the region of interest is provided in addition to the recognition result.

It is preferable that the classification result is acquired using the first medical image and a plurality of medical images in a temporal vicinity of the first medical image acquired at an earlier time point than the second medical image.

It is preferable that the recognizer is selected according to the classification result and a selection history of the recognizer at a previous time point.

It is preferable that weighting is performed on the classification results of the plurality of medical images in the temporal vicinity based on the selection history, and the recognizer is selected using the weighted classification results.

It is preferable that the classification result in which a degree of certainty is calculated for each imaged part is acquired from the classifier, and in a case in which the degree of certainty is less than a specific reference value, use of the recognizer at the previous time point from the selection history is continued.

It is preferable that each of the plurality of recognizers is a trained deep learning model trained on a different data set.

It is preferable that, in a case in which the classification result is a first part, the recognizer trained on a data set including more images captured at the first part than images captured at a part different from the first part is selected.

It is preferable that each of the plurality of recognizers has a function of recognizing different regions of interest, and the selected recognizer acquires the recognition result by executing at least one of position detection, type determination, or region measurement for the region of interest in the medical image.

It is preferable that the medical image processing apparatus may further comprise a discriminator in which the classifier and the plurality of recognizers are integrated, the discriminator calculating an intermediate feature amount in the medical image, classifying the imaged part from the intermediate feature amount, and recognizing the region of interest from the intermediate feature amount and the imaged part, in which a first intermediate feature amount calculated from the first medical image and a second intermediate feature amount calculated from the second medical image are acquired, the classification result based on the first intermediate feature amount is acquired, a deep learning model corresponding to the classification result in the first intermediate feature amount from a plurality of deep learning models is selected, and the recognition result of the region of interest in the second intermediate feature amount from the selected deep learning model is acquired.

According to another aspect of the present invention, there is provided an endoscope system comprising: the medical image processing apparatus; and an endoscope that acquires the medical image.

According to the aspects of the present invention, it is possible to achieve both selecting an optimal examination support model for each part of a medical image and performing real-time processing.

### BRIEF DESCRIPTION OF THE DRAWINGS

FIG. 1 is an explanatory diagram showing connection apparatuses of a medical image processing apparatus.

FIG. 2 is an explanatory diagram of a time series medical image group acquired by a medical image processing apparatus.

FIG. 3 is a block diagram showing a function of the medical image processing apparatus.

FIG. 4 is an explanatory diagram of a medical image to be subjected to part classification.

FIG. 5 is an explanatory diagram of a medical image in which region-of-interest recognition is performed using a classification result.

FIGS. 6A and 6B are explanatory diagrams of a case in which series processing is performed and a case in which parallel processing is performed in part classification and region-of-interest recognition.

FIG. 7 is an explanatory diagram of parallel processing of part classification and region-of-interest recognition using a classification result at a previous time point.

FIG. 8 is an explanatory diagram of a method for selecting a recognizer in a second selection mode.

FIG. 9 is an explanatory diagram of a method for selecting a recognizer in a third selection mode.

FIG. 10 is an explanatory diagram of a method for selecting a recognizer in a fourth selection mode.

FIG. 11 is an explanatory diagram of parallel processing of part classification, region-of-interest recognition using a classification result at a previous time point, and image creation using a recognition result at the previous time point.

FIG. 12 is an explanatory diagram in which parallel processing of part classification, region-of-interest detection, and image creation is performed for each frame.

FIG. 13 is an image diagram in which a display image is displayed.

FIG. 14 is an explanatory diagram for displaying a schema diagram on a screen.

FIG. 15 is a flowchart showing a series of flow of processing of the present invention.

FIG. 16 is a block diagram showing a function of a medical image processing apparatus in a second embodiment.

FIG. 17 is an explanatory diagram of parallel processing of performing part classification, region-of-interest detection, and display image creation in the second embodiment.

### DESCRIPTION OF THE PREFERRED EMBODIMENTS

### First Embodiment

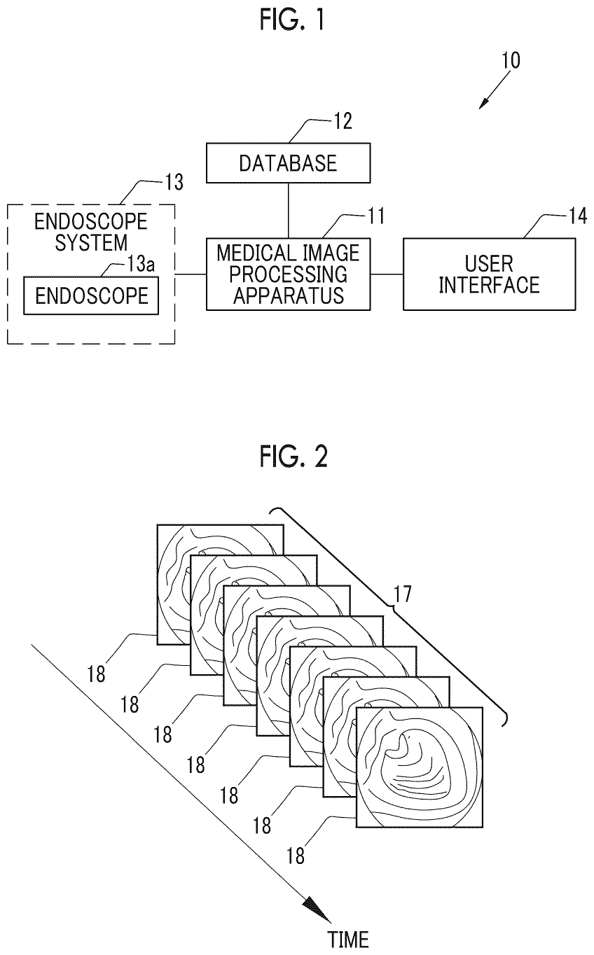

FIG. 1 is a diagram showing connection apparatuses of a medical image processing apparatus 11 in a medical image processing system 10 according to an embodiment of the present invention. The medical image processing system 10 includes a medical image processing apparatus 11, a database 12, an endoscope system 13 including an endoscope 13a, and a user interface (UI) 14. The medical image processing apparatus 11 is electrically connected to the database 12, the endoscope system 13, and the user interface 14. Further, instead of the medical image processing system 10, the endoscope system 13 may be configured to include the medical image processing apparatus 11.

The database 12 is an apparatus capable of storing the acquired image and transmitting and receiving data to and from the medical image processing apparatus 11 and may be a recording medium such as a universal serial bus (USB) or a hard disc drive (HDD).

The user interface 14 may be a device, such as a touch panel, in which a notification unit and an input unit are integrated, or a device, such as a display, a keyboard, or a mouse, in which a notification unit and an input unit are different from each other. Further, all of these devices may be included. In addition to or instead of the keyboard and the mouse, the input unit in the user interface 14 may perform input using an input unit provided in the medical apparatus, such as a foot pedal, a voice recognizer, or a switch on the endoscope 13a.

As shown in FIG. 2, the medical image processing apparatus 11 acquires a medical image group 17 captured by a medical examination using the endoscope system 13 or other medical apparatuses. The medical image group 17 is a time-series image composed of a plurality of temporally continuous medical images 18. The medical image 18 is a frame constituting a moving image or a still image. In imaging of the medical examination, unless otherwise particularly designated, white light is used as illumination light, video signals of 60 frames per second (60 fps) are acquired, and an imaging time is recorded. Additionally, it is preferable to measure the time point in units of one-hundredth of a second in a case in which the video signal is at 60 fps.

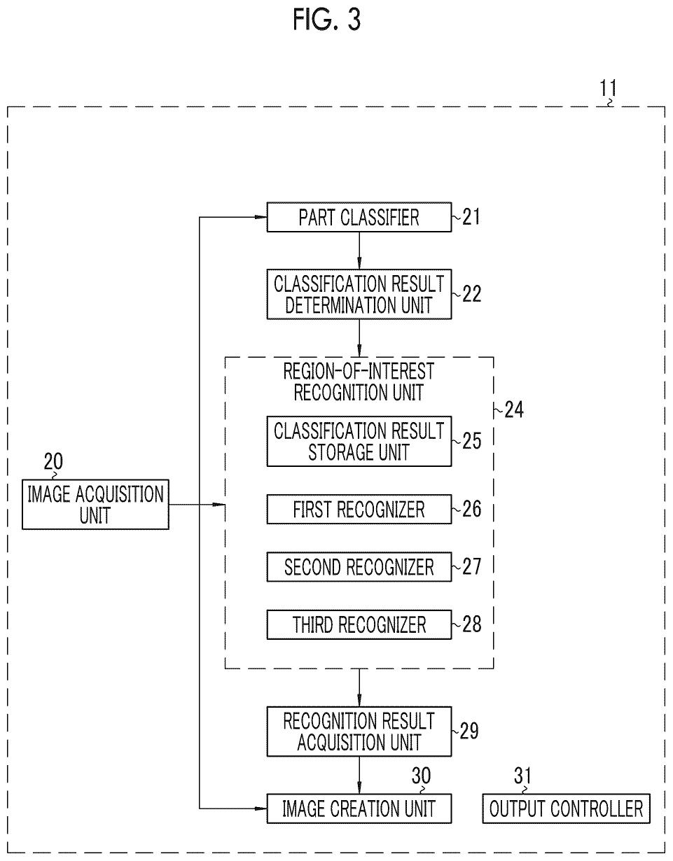

As shown in FIG. 3, in the medical image processing apparatus 11, a program in a program memory is operated by a central control unit (not shown) constituted by a processor, and whereby the functions of an input receiving unit (not shown), an image acquisition unit 20, a part classifier 21, a classification result determination unit 22, a region-of-interest recognition unit 24, a recognition result acquisition unit 29, an image creation unit 30, and an output controller 31 are implemented and controlled. In addition, the region-of-interest recognition unit 24 includes a classification result storage unit 25, a first recognizer 26, a second recognizer 27, and a third recognizer 28.

Since a lesion or an organ greatly differs depending on an imaged part, the region-of-interest recognition unit 24 includes a trained model optimized for each part. By switching the trained model for each part according to the imaged part, it is possible to always provide support by a high-performance trained model. Therefore, the medical image processing apparatus 11 comprises a part classifier 21 that classifies the imaged part in the medical image 18 and a plurality of recognizers that recognize a region of interest R in the medical image 18 and correspond to each imaged part.

The imaged parts classified by the part classifier 21 are associated with the types of imaged parts learned by each recognizer. The correspondence relationship may be configured such that they match, or may be configured such that in a case in which the classification result is “stomach fundus” or “gastric body”, a recognizer trained on “stomach” is selected. Accordingly, it is possible to provide examination support such as detection, discrimination, and measurement of a lesion for region-of-interest recognition with higher accuracy than in a case in which a plurality of parts during the same examination are covered by the same trained model.

The input receiving unit receives an input from a user via the user interface 14. In the medical image processing apparatus 11, a program related to processing such as image processing is stored in the program memory (not shown).

The image acquisition unit 20 acquires the medical image 18 which is a frame image from the endoscope system 13 or the like. Every time the medical image 18 is acquired, the medical image 18 is input to the part classifier 21, the region-of-interest recognition unit 24, and the image creation unit 30 to execute part classification, region-of-interest recognition, and image editing.

The part classifier 21 has a function of a trained model necessary for classification processing of a part such as an organ or a tissue of a living body captured by the medical image 18, and executes part classification processing one by one. The part classifier 21 includes a convolutional neural network (CNN), which is a computer algorithm consisting of a neural network that performs machine learning, and is trained in advance using data covering parts that may be imaged during upper endoscopy, such as the esophagus, stomach, duodenum, nasal cavity, oral cavity, and pharynx, and infers the part classification in the input medical image 18 and calculates the degree of certainty of the classified parts. The inferred part and its degree of certainty are used as a classification result.

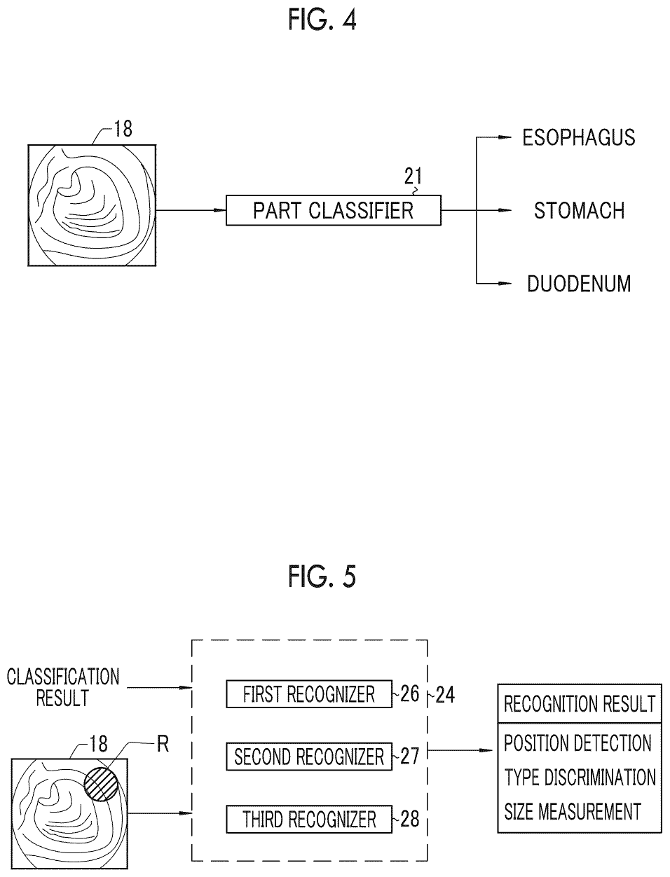

As shown in FIG. 4, in a case in which the medical image 18 is input to the part classifier 21, classification processing is performed for the part to be classified to which the medical image 18 corresponds using inference by machine learning, and the imaged part is inferred. For example, in a case of observing the upper gastrointestinal tract with an endoscope, the inferred parts include the esophagus, stomach, and duodenum in the medical image 18. Furthermore, the degree of certainty corresponding to the inferred part is calculated, and the classification result having the imaged part and the degree of certainty is transmitted to the classification result determination unit 22.

The degree of certainty is an indicator that indicates reliability of a classification result calculated by inference of a learning model using a match rate with learning contents learned in advance, an erroneous detection rate in each classification target, image quality information, or the like, and for example, is represented using a percentage (%).

The classification result determination unit 22 acquires, from the part classifier 21, a classification result in which the degree of certainty is calculated for each imaged part, and determines whether or not the degree of certainty is equal to or greater than a specific reference value such as a preset threshold value. The degree of certainty is calculated from a match rate or the like with the learned data set in the classification by the part classifier 21. In a case in which the degree of certainty is equal to or greater than the specific reference value, the classification result can be transmitted to the region-of-interest recognition unit 24 and used to select the recognizer; however, in a case in which the degree of certainty is less than the specific reference value, the classification result is changed to “unclassifiable” and output to the region-of-interest recognition unit 24.

The region-of-interest recognition unit 24 determines a recognizer to be used for region-of-interest recognition processing from the first recognizer 26, the second recognizer 27, and the third recognizer 28 based on the classification result output from the classification result determination unit 22, and executes region-of-interest recognition on the input medical image 18 using the recognizer. A classification result stored in the classification result storage unit 25 at a previous time point may be used to determine the recognizer. Alternatively, a recognizer selection history for a certain period of time may be stored and a past selection history may be used for recognizer selection.

The classification result storage unit 25 temporarily stores the classification results acquired by the region-of-interest recognition unit 24 from the part classifier 21. The stored classification results are used to select a recognizer according to a selection mode, which will be described later. The temporary storage is preferably performed in a storage memory (not shown) implemented in the medical image processing apparatus. Further, the database 12 may have this function instead of the storage memory.

Each of the recognizers includes a convolutional neural network (CNN), which is a computer algorithm consisting of a neural network that performs machine learning, and recognizes a specific subject captured in the input medical image 18 according to the content of learning of each part in the living body carried out in advance. Diagnostic support through recognition of regions of interest includes lesion detection, lesion discrimination, organ detection, and lesion measurement. Each recognizer has the function of a trained deep learning model trained on a different data set.

A data set including a larger number of images captured at the corresponding imaged part than images captured at other imaged parts to be classified by the part classifier 21 is used for the learning in each recognizer. For example, a recognizer that performs the region-of-interest recognition with respect to a medical image of “esophagus” is a recognizer that is trained on a data set including more images captured from “esophagus” than images captured from “stomach” and “duodenum”, which are parts different from “esophagus”. It is preferable that each recognizer performs learning using a data set composed of images captured at the part to be classified by the part classifier 21.

As shown in FIG. 5, the region-of-interest recognition unit 24 receives inputs of the classification result from the classification result determination unit 22 and the medical image 18 from the image acquisition unit 20, and selects a recognizer to be used for recognition processing of the medical image 18 according to the classification result. In the region-of-interest recognition, the presence or absence of the region of interest R such as a specific subject of each input medical image 18 is determined. Further, in addition to the determination of the presence or absence, it is preferable that each recognizer has different functions of recognizing the region of interest, and the selected recognizer executes at least one of position detection, type determination, or size measurement of the region of interest R to acquire a recognition result. The specific subject recognized as the region of interest R is a subject having a high likelihood of a lesion, a treatment scar, or the like.

Specifically, each recognizer has at least one of a position detection function, a type determination function, or a size measurement function depending on the corresponding imaged part. The position detection function is a function of detecting a position in the organ or the image with respect to the region of interest R, the type determination function is a function of discriminating whether or not the region of interest R is a lesion or a type such as a tumor or a hemorrhage, and the size measurement function is a function of measuring a size, such as the total length, of the region of interest R. For example, the position detection function of the region of interest R is operated in a recognizer corresponding to “esophagus”, and the type determination function of the region of interest is also operated in a recognizer corresponding to “stomach”.

Four or more types of recognizers may be provided, and in that case, classification is performed such that the classification result by the part classifier 21 corresponds to each recognizer. For example, there is a recognizer that has learned about “nasal cavity”, “oral cavity”, and “pharynx”. In addition, different recognizers may be provided for “stomach fundus”, “gastric body”, and “antrum” instead of the “stomach”, and as the part classifier 21, a classifier trained to classify “stomach fundus”, “gastric body”, and “antrum” instead of “stomach” may be used.

The recognition result acquisition unit 29 acquires a recognition result of each recognition, for example, information such as the position, the type, or the size of the region of interest R. The recognition result includes at least a discrimination result of the presence or absence of the region of interest R in the medical image 18 and a type of a recognizer used.

The image creation unit 30 acquires a recognition result and a classification result, and creates a display image. The display image is created by superimposing and displaying the recognition result or the classification result on the medical image 18 and the schema diagram. The created display image is transmitted to the output controller 31. The classification result is that of the medical image 18 at the timing used to select the recognizer.

The output controller 31 performs control to display, on the screen, notification of the classification result and the recognition result such as the display of the display image created by the image creation unit 30. In addition to image display, voice notification may also be provided. In a case in which the display image is displayed, the medical image 18 may be displayed on the screen at the same time, or only the display image may be displayed. In a case in which voice notification is provided without displaying the display image, the medical image group 17 is displayed.

The medical image group 17 acquired by the image acquisition unit 20 from the database 12 or the endoscope system 13 is subjected to part classification processing and recognition processing one by one as the medical image 18. By using a recognizer that has been trained on the classified imaged parts, it is possible to perform region-of-interest recognition with high accuracy.

On the other hand, in the classification or recognition of the image using the trained model, it is necessary to reduce the model size in order to shorten a processing time, and as a result, the accuracy is reduced. Since the medical image processing apparatus 11 performs real-time part classification and region-of-interest recognition of the medical image group 17, it is necessary to secure a processing time for maintaining sufficient accuracy for both.

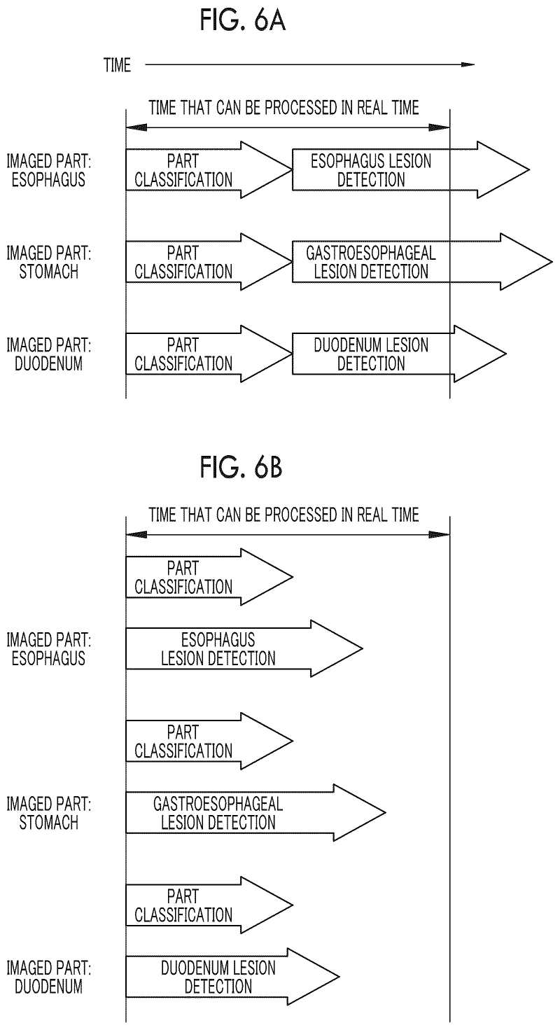

In FIGS. 6A and 6B, as shown in FIG. 6A, in series processing in which the region-of-interest recognition processing is performed on the medical image 18 subjected to the part classification processing, in a state in which certain accuracy is maintained, the sum of the processing time by the part classifier 21 and the processing time by the recognizer may not fit within the time that can be processed in real time, and it may not be possible to achieve both accuracy and real-time processing. Therefore, as shown in FIG. 6B, by selecting a recognizer corresponding to the classification result of the previous frame, part classification in the medical image 18 by the part classifier 21 and region-of-interest recognition in the medical image 18 by one of the recognizers are performed in parallel processing.

In the case of an endoscope or ultrasound diagnosis, since examinations are continuously performed, switching of parts does not occur frequently in units of frames. Therefore, it can be said that there is almost no negative effect even in a case in which the part recognition result of the previous frame is applied to the current frame. Note that in a case in which each recognizer randomly performs functions, such as position detection, size measurement, and type discrimination, with respect to a region of interest in recognition processing, differences may occur in the required processing time.

By performing the parallel processing of the part classification and the region-of-interest recognition, the processing time of the part classification and the processing time of the region-of-interest recognition are individually secured, and therefore a high-performance trained model can be used for the part classification and the region-of-interest recognition.

It is preferable that a notification of the recognition result is provided using screen display, voice, or the like. In real-time observation, as long as a notification only distinguishes the presence or absence of the region of interest without image creation, such as only issuing an alert at the time when the region of interest is recognized, the processing may be performed continuously with the region-of-interest recognition. On the other hand, in a case of providing a notification of a specific recognition result of the region of interest, in addition to part classification by the part classifier 21 and region-of-interest recognition by the region-of-interest recognition unit 24, image editing by the image creation unit 30 are performed in parallel processing.

Regarding examples of the present embodiment, in a first example, parallel processing of providing a notification of only the presence or absence of a region of interest in a recognition result will be described, and in a second example, parallel processing of performing image display of the recognition result will be described. In the first example and the second example, it is assumed that the first recognizer 26 is a recognizer corresponding to the classification result of “esophagus”, the second recognizer 27 is a recognizer corresponding to the classification result of “stomach”, and the third recognizer 28 is a recognizer corresponding to the classification result of “duodenum”.

A first example will be described. In the first example, only the presence or absence of a region of interest is output as a recognition result during real-time observation. The recognition result by the recognizer is notified to the user within the real-time processing time. The notification method is, for example, a sound such as a beep sound that takes a short processing time, or a screen display of fixed symbols or characters.

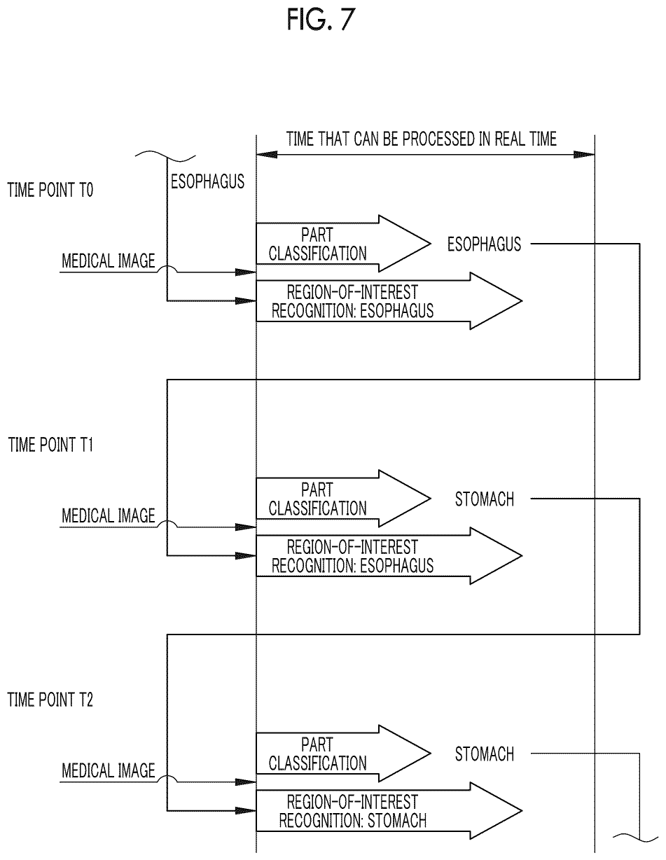

As shown in FIG. 7, in the parallel processing performed at any time point T1, the classification result performed on the medical image 18 at time point T0 is used at time point T1. Therefore, the image acquisition unit 20 acquires a medical image group 17 that is a time-series medical image including a medical image 18 at time point T0 and a medical image 18 at time point T1 acquired at a later time point than the medical image 18 at time point T0. From time point T0 to time point T1, the region-of-interest recognition unit 24 acquires the classification result of the imaged part in the medical image 18 at time point T0 from the part classifier 21, selects a recognizer corresponding to the classification result of the medical image 18 at time point T0 from the first recognizer 26, the second recognizer 27, or the third recognizer 28, and executes region-of-interest recognition. The recognition result of the region of interest in the medical image 18 at time point T1 is acquired from the selected recognizer. The acquired recognition result is transmitted to the recognition result acquisition unit 29.

For example, in a case in which the classification result determination unit 22 acquires a classification result of “esophagus” in the part classification at time point T0, at time point T1, which is the timing at which parallel processing is performed after time point T0, the first recognizer 26 is selected for region-of-interest recognition, and region-of-interest recognition for the medical image 18 at time point T1 is performed. Furthermore, the part classification of the medical image 18 at time point T1 is performed in parallel processing.

In a case in which the classification result determination unit 22 acquires the classification result of “stomach” in the part classification at time point T1, the second recognizer 27 is selected as the recognizer at time point T2, which is the timing at which parallel processing is performed after time point T1. In a case in which the same classification result is obtained continuously, such as the classification result of “stomach” even in the part classification at time point T2, the region-of-interest recognition unit 24 may continue the selection situation instead of resetting the selection situation and selecting again.

The classification result determination unit 22 acquires, from the part classifier 21, a classification result in which the degree of certainty is calculated for each imaged part, and discriminates whether or not the degree of certainty is equal to or greater than a specific reference value such as a preset threshold value. In a case in which the degree of certainty is less than the specific reference value, a classification result of “unclassifiable” is transmitted to the region-of-interest recognition unit 24 instead of the inferred imaged part. In a case in which the region-of-interest recognition unit 24 acquires a classification result of “unclassifiable”, the region-of-interest recognition is performed by continuing to use the recognizer selected at the previous time point from a recognizer selection history.

Regarding the selection of a recognizer, the recognizer may be selected not only by using a classification result acquired in the immediately previous parallel processing but also by using classification results at a plurality of previous time points. For example, the classification result determination unit 22 includes first to fourth selection modes and the respective selection modes execute select recognizers using different rules. It is preferable that the user can randomly switch between the modes. Further, the contents of each selection mode may be combined and executed.

As described above, in the first selection mode, a recognizer is selected using the classification result obtained by performing a determination of a degree of certainty on the medical image 18 in the immediately preceding parallel processing.

In the second selection mode, a classification result is determined using the medical image 18 subjected to parallel processing and a plurality of medical images 18 in the temporal vicinity thereof. In a case in which parallel processing is performed at time point T1, the classification results of the medical image 18 subjected to parallel processing at time point T0 and the temporarily stored medical image 18 acquired within a certain range from time point T0 are integrated. Note that all of the plurality of medical images 18 used for selecting a recognizer were acquired at a timing earlier than the medical image 18 at time point T1 used for parallel processing.

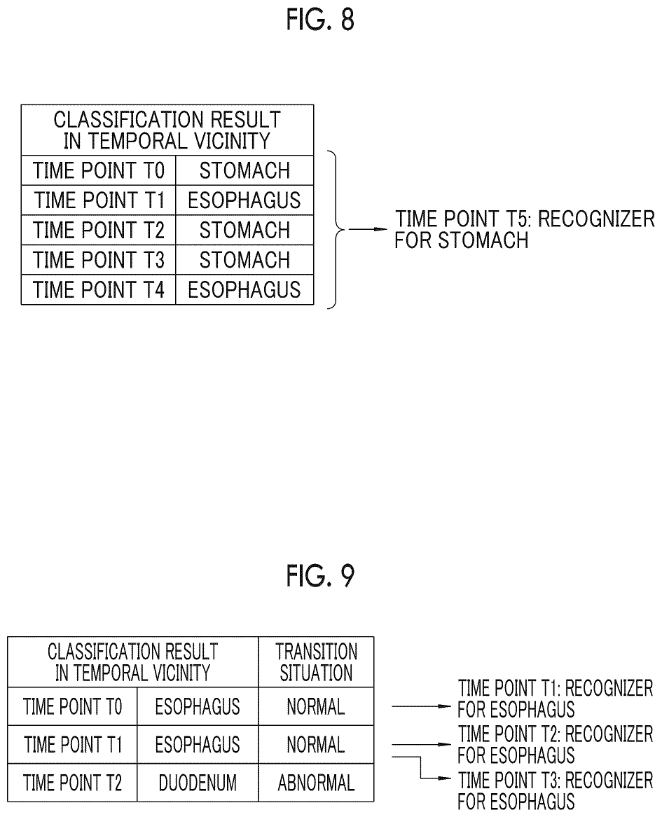

As shown in FIG. 8, in a case in which the classification results of the latest five times are integrated, the classification result storage unit 25 stores the classification results of time points T0 to T4 at time point T5, and the region-of-interest recognition unit 24 uses the most common classification result as an integrated result for selecting a recognizer. Even in a case in which there is a classification result of “esophagus” at time point T4, which is the immediately preceding time point, in a case in which “stomach” is the majority, the second recognizer 27 is selected. As for the classification results of the temporal vicinity, at least three times in which a majority vote is possible are used. The integrated result may be acquired by averaging the CNN results or removing outliers instead of using a majority vote based on a plurality of classification results. By integrating and using the results of a plurality of frames, classification can be performed with higher accuracy.

In the third selection mode, a recognizer is selected according to the acquired classification result of the medical image 18 and the recognizer selection history at the previous time point. By comparing the recognizer selection history in the previous parallel processing with the imaged parts of the acquired classification result and determining whether the transitions in the classification results of the imaged parts are natural or unnatural in terms of the human body structure, classification results with a high likelihood of misclassification are specified. In the upper gastrointestinal tract, the order of at least the human body structures of “esophagus”, “stomach”, and “duodenum” is stored.

As shown in FIG. 9, for example, in a case in which the “esophagus” is continuously detected in the parallel processing up to time point T1, the transition situation is determined to be normal in the parallel processing up to time point T2, and the first recognizer 26 corresponding to the imaged part of “esophagus” is selected. Regarding the parallel processing at time point T2, in a case in which the classification result of “duodenum” is acquired, it is determined that this is an abnormal state with a high likelihood of misclassification, and in selecting the recognizer to be used at time point T3, the classification result of “duodenum” is not used, and the second recognizer 27, which is the previous selection result, is continued to be used. It is possible to prevent the selection of the wrong recognizer, a decrease in the accuracy of region-of-interest recognition due to the wrong recognizer, and the occurrence of unexpected behavior. By excluding classification results with a high likelihood of misclassification, an appropriate recognizer is selected and highly accurate region-of-interest recognition is performed.

In the fourth selection mode, the classification results of the plurality of medical images 18 in the temporal vicinity are weighted based on the recognizer selection history at the previous time point, and the recognizer is selected from the integrated result of integrating the weighted classification results. For example, a score is calculated by multiplying the number of detections of each classification result in a certain period of time by a weighting coefficient. To perform weighting, two medical images 18 in the temporal vicinity may be used.

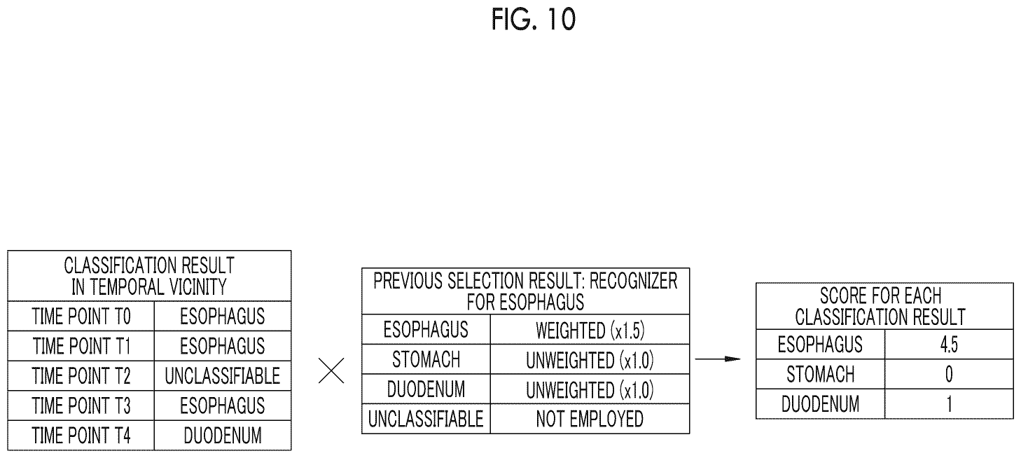

As shown in FIG. 10, in a case in which the previous selection result is the second recognizer 27, the classification results for parts that do not correspond to “esophagus” are given the same size, and the parts corresponding to “esophagus” are weighted to be 1.5 times as large to calculate the score. Note that classification results of “unclassifiable” are not employed in score calculation. The part with the highest total calculated score is set as the integrated result, and a recognizer corresponding to the integrated result is selected. In a case in which the classification results are “esophagus” three times, “duodenum” once, and “unclassifiable” once at time points T0 to T4, the score is 4.5 for the esophagus and 1 for the duodenum, and the second recognizer 27 is selected for parallel processing at time point T5.

A second example will be described. In the second example, a display image is created in real time and a notification is provided by displaying the display image on a screen. In examination using an endoscope, by creating display images that can support diagnosis and treatment in real time, it is possible to determine the necessity of treatment during the same examination.

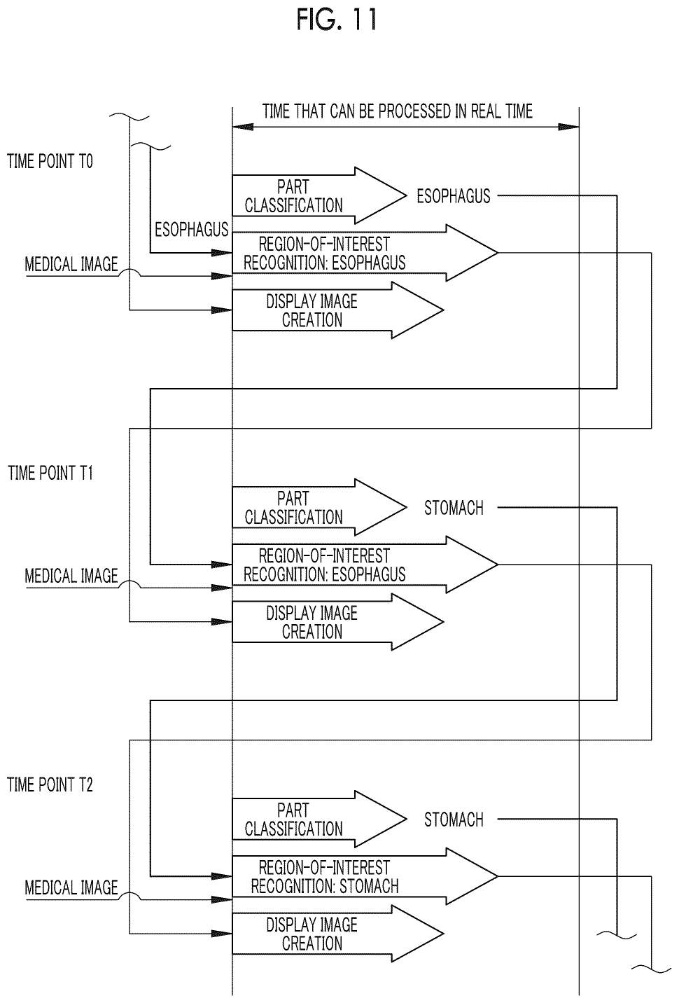

As shown in FIG. 11, in a case in which part classification, region-of-interest recognition, and display image creation are performed in parallel processing, the region-of-interest recognition uses the classification result of the immediately previous timing, and the display image creation uses the recognition result of the immediately previous timing based on the classification result of the two previous timing. Specifically, in the parallel processing at time point T2, the display image 40 is created by superimposing, on the medical image 18 at time point T2, the classification of the imaged part in the medical image 18 at time point T2 by the part classifier 21, recognition of the region of interest in the medical image 18 at time point T2 using a recognizer based on the imaged part in the medical image 18 at time point T1, and the recognition result in the medical image at time point T1.

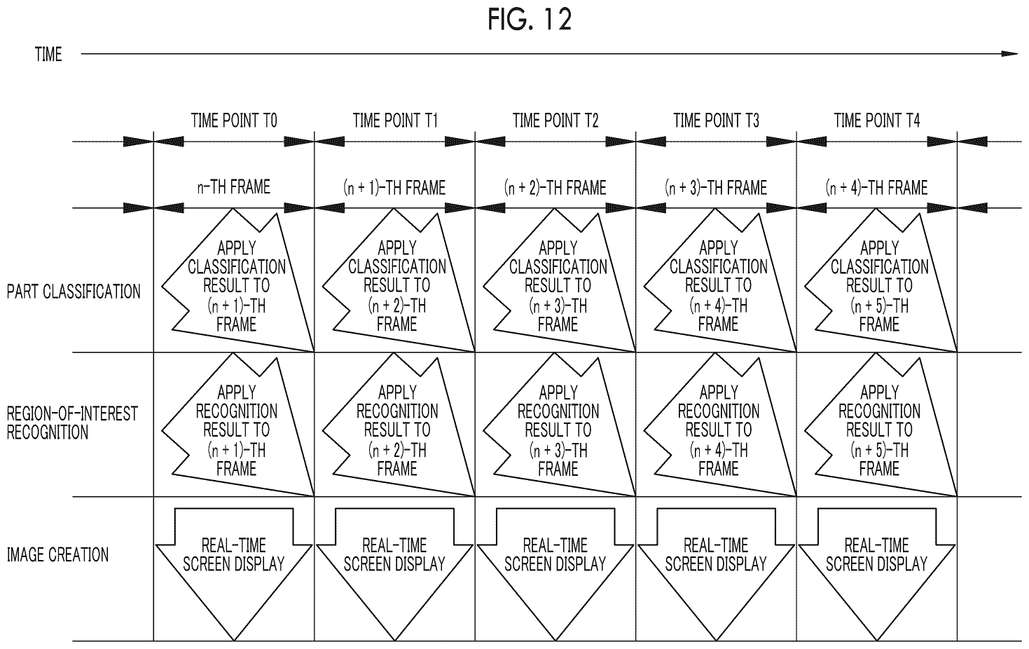

As shown in FIG. 12, in a case in which parallel processing is performed frame by frame on a plurality of medical images 18 included in the medical image group 17, the image is created using the recognition result of the immediately previous frame using the classification result of the two previous frame. Parallel processing for acquiring the medical images 18 of the n-th frame at time point T0, the (n+1)-th frame at time point T1, and the (n+2)-th frame at time point T2 will be described. In the parallel processing at time point T2, the part classifier 21 performs part classification of the (n+2)-th frame, a recognizer based on the classification result of the (n+1)-th frame performs region-of-interest recognition of the (n+2)-th frame, and using the recognition result of the (n+1)-th frame, the image creation unit 30 edits the (n+2)-th frame to create a display image. The created display image is displayed on the screen in real time.

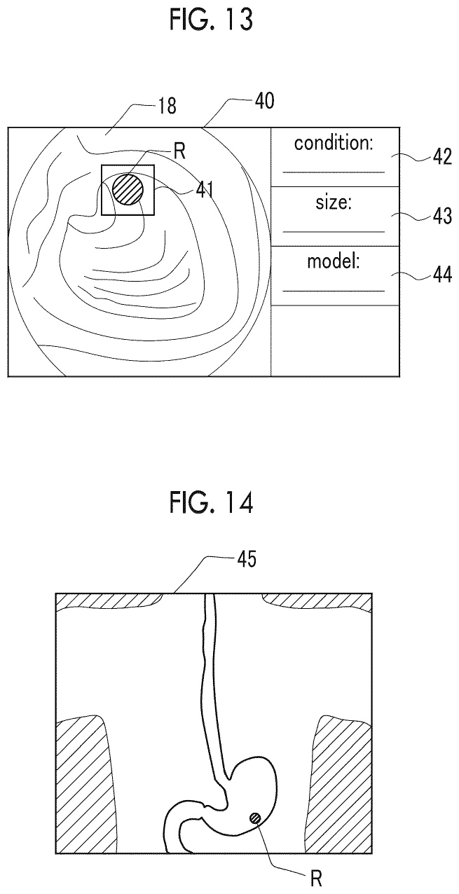

As shown in FIG. 13, the display image 40 created by the image creation unit 30 based on the recognition result is used for image display. In the display image 40, display or non-display of a marker 41, a type display field 42, and a size display field 43 is determined as appropriate depending on the preset settings and the functions implemented by the recognizer. A used recognizer display field 44 displays the selection result of the recognizer used for region-of-interest recognition.

The marker 41 that emphasizes the position of the region of interest R is displayed to be superimposed on the medical image 18, and the type display field 42 displays whether or not the region of interest R is a lesion in a case in which discrimination of the region of interest R is performed by the recognizer, and in a case in which it is a lesion, the type of lesion. The size display field 43 displays the measurement results obtained in a case in which the region of interest R is measured by the recognizer. In the used recognizer display field 44, the type of recognizer used to recognize the region of interest R is displayed.

The type display field 42, the size display field 43, and the used recognizer display field 44 may be displayed to be superimposed on the medical image 18, or may be displayed next to the region of the medical image 18 without being superimposed.

As shown in FIG. 14, a schema diagram 45 in which information on the region of interest R is displayed in a superimposed manner may be created and displayed on the screen together with the display image 40. The schema diagram 45 is a schematic diagram of a biological range of an examination target, and displays, for example, the range from the esophagus to the duodenum. In the schema diagram 45, it is possible to notify at which position in the subject's body the region of interest R has been detected. furthermore, in a case in which a plurality of regions of interest R are detected, the positions of the respective regions of interest R may be displayed at the same time.

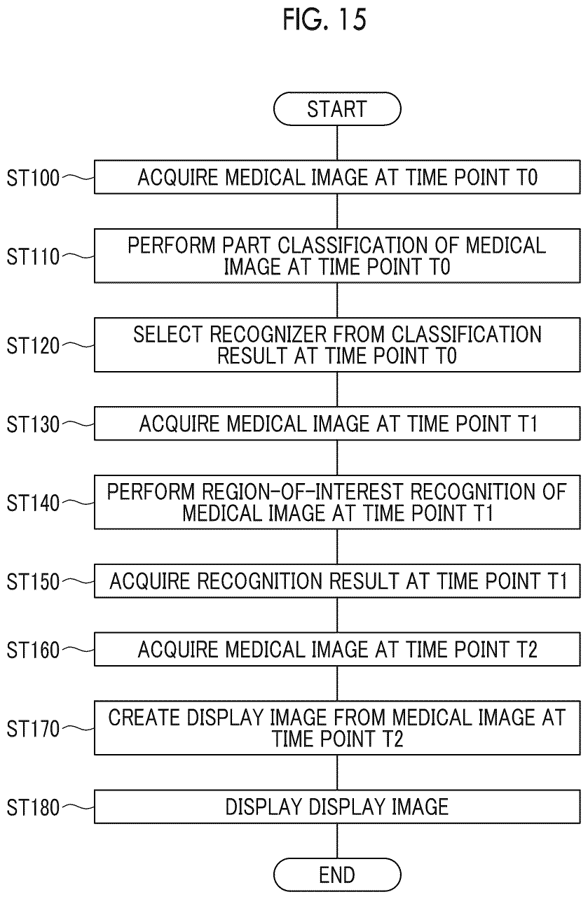

A series of flows of medical image processing according to the present embodiment will be described along the flowchart shown in FIG. 15. The medical image processing apparatus 11 acquires the medical image 18 at time point T0 that constitutes the medical image group 17 captured by the medical examination (Step ST100). The acquired medical image 18 is input to the part classifier 21 and part classification is performed (Step ST110). The acquired classification results are transmitted to the region-of-interest recognition unit 24, and a recognizer corresponding to the classification results is selected (Step ST120).

The medical image 18 at time point T1, which is the next timing after time point T0 to be input into the selected recognition, is acquired (Step ST130). The region-of-interest recognition of the medical image 18 at time point T1 is performed using the selected recognizer (Step ST140). The acquired recognition result is transmitted to the image creation unit 30 (Step ST150).

The image creation unit 30 acquires the medical image 18 at time point T2, which is the next timing after time point T1, to be used to create the display image 40 of the recognition result (Step ST160). The display image 40 is created using the medical image 18 at time point T2 and the recognition result (Step ST170). The created display image 40 is displayed on the screen and thereby a notification of the recognition result is provided (Step ST180).

With the above contents, by performing parallel processing on part classification and regions of interest, it is possible to achieve both selecting a recognizer that is the optimal examination support model for each part and performing real-time processing.

### Second Embodiment

In the first embodiment, an aspect has been described in which the selection of the recognizer is controlled using the part classification result obtained by classifying each medical image 18 from the part classifier 21 in parallel processing. In contrast, an aspect will be described in which a determiner in which the functions of the part classifier 21 and a plurality of recognizers corresponding to each imaged part are integrated is provided and the calculation of an intermediate feature amount is executed as a common process. Note that descriptions of other contents common to the above embodiments will be omitted.

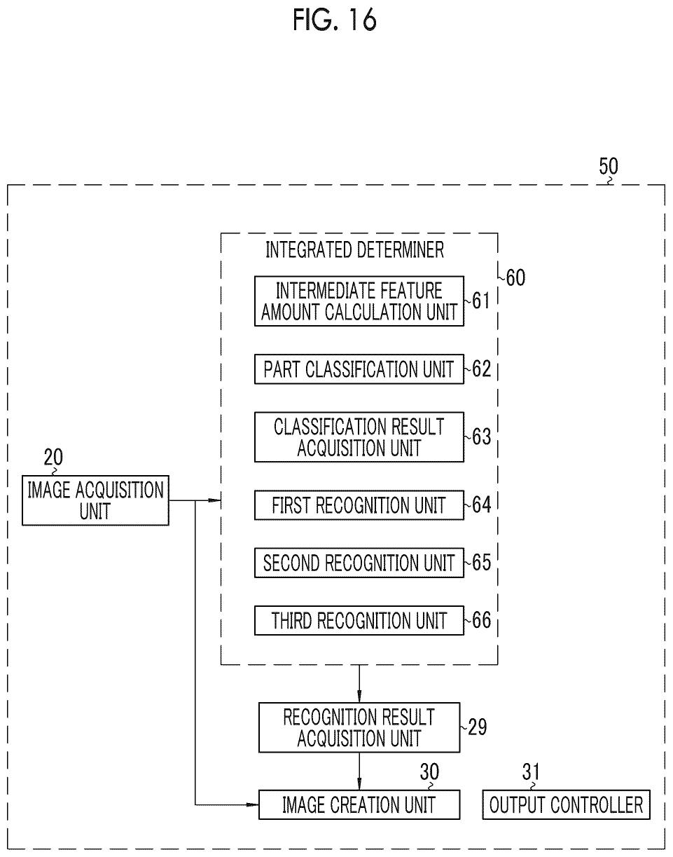

As shown in FIG. 16, a medical image processing apparatus 50 according to the second embodiment has functions of an image acquisition unit 20, an integrated determiner 60, a recognition result acquisition unit 29, an image creation unit 30, and an output controller 31. The integrated determiner 60 performs parallel processing of the part classification and the region-of-interest recognition on the medical image 18, instead of the part classifier 21, the classification result determination unit 22, and the region-of-interest recognition unit 24 of the first embodiment.

The integrated determiner 60 has a function in which an intermediate feature amount calculation unit 61, a part classification unit 62, a classification result storage unit 25, a first recognition unit 64, a second recognition unit 65, and a third recognition unit 66 are integrated. The integrated determiner 60 calculates an intermediate feature amount with the intermediate feature amount calculation unit 61 for the medical image 18 transmitted from the image acquisition unit 20, and transmits the calculated intermediate feature amount to either the part classification unit 62 or the selected recognition unit.

The intermediate feature amount calculation unit 61 calculates intermediate feature amounts of the medical image 18, which are common in the part classification and the region-of-interest recognition. The calculated intermediate feature amount is transmitted to the part classification unit 62 and is transmitted to any one of the recognition units selected by the classification result acquisition unit 63.

The part classification unit 62 performs part classification processing on the acquired intermediate feature amounts. It is preferable that the part classification unit 62 has the function of the part classifier 21 of the first embodiment, and receives an input of intermediate feature amounts instead of the medical image 18 and executes part classification.

The classification result acquisition unit 63 acquires the classification result by the part classification unit 62 and selects a recognition unit that performs region-of-interest recognition corresponding to the classification result from any one of the first recognition unit 64, the second recognition unit 65, and the third recognition unit 66. The intermediate feature amount is transmitted to the selected recognition unit. Further, the functions of the classification result determination unit 22 and the classification result storage unit 25 in the first embodiment may be provided, and the first to fourth selection modes may be switched and executed.

In a case in which the first recognition unit 64 is selected by the classification result acquisition unit 63, the first recognition unit 64 performs region-of-interest recognition in the transmitted intermediate feature amount. It is preferable that the first recognition unit 64 has the function of the first recognizer 26 of the first embodiment, and receives an input of intermediate feature amounts instead of the medical image 18 and executes region-of-interest recognition.

Similarly to the first recognition unit 64, it is preferable that the second recognition unit 65 has the function of the second recognizer 27 and the third recognition unit 66 has the function of the third recognizer 28, and the second recognition unit 65 and the third recognition unit 66 receive an input of intermediate feature amounts instead of the medical image 18 and execute region-of-interest recognition. Note that each recognition unit may be provided with a deep learning model that corresponds to each part and performs recognition processing from intermediate feature amounts.

In parallel processing, the integrated determiner 60 acquires classification results based on the intermediate feature amounts in the previous parallel processing from the part classification unit 62, and uses the classification results for region-of-interest recognition of the intermediate feature amounts in the acquired medical image 18. A recognition unit corresponding to the classification result in the previous parallel processing is selected from each recognition unit, and a recognition result of the region of interest in the intermediate feature amount is acquired from the selected recognition unit.

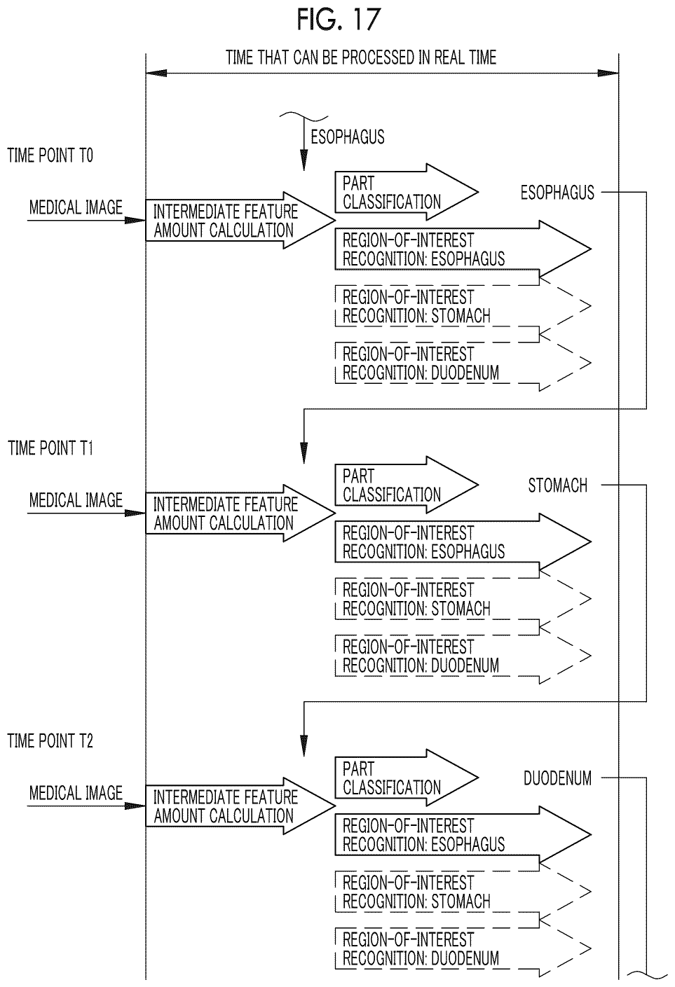

As shown in FIG. 17, in the parallel processing at time point T1, part classification and region-of-interest recognition are performed using the classification results classified from the medical image 18 at time point T0 and the intermediate feature amounts calculated from the medical image 18 at time point T1. Further, the recognition result obtained by the region-of-interest recognition is transmitted to the recognition result acquisition unit 29. For example, the integrated determiner 60 acquires the classification result of “esophagus” for the intermediate feature amount at time point T0 by the time of the intermediate feature amount calculation at time point T1, and selects the first recognition unit 64, which is a recognition unit corresponding to “esophagus”, which is the classification result of the intermediate feature amount at time point T0, from the plurality of recognition units. The intermediate feature amount at time point T1 is transmitted to the part classification unit 62 and the first recognition unit 64, which is a recognition unit corresponding to “esophagus”, to execute part classification and region-of-interest recognition, and acquire classification results and recognition results of “stomach”.

Similarly, in the parallel processing at time point T2, the classification result of “stomach” classified at time point T1 is transmitted to the classification result acquisition unit 63 by the time of intermediate feature amount calculation of the medical image 18 at time point T2, and the second recognition unit 65 corresponding to “stomach” is selected and parallel processing is performed.

As described above, even before acquiring the part classification results, part of the processing of part classification and region-of-interest recognition of the medical image 18 at each time point can be started. Therefore, compared to the configuration of the first embodiment, it is possible to secure a longer time for parallel processing, particularly for region-of-interest recognition. Alternatively, since the time from acquiring the classification result to ending the region-of-interest recognition can be shortened, real-time processing of the medical image group 17 at a higher frame rate than in the first embodiment is possible.

Note that the medical image processing system 10 may be realized including a medical image processing apparatus that has both the functions of the part classifier 21, the classification result determination unit 22, and the region-of-interest recognition unit and the function of the integrated determiner 60, and uses different functions as necessary.

In the above embodiments, the example in which the medical image processing apparatus 11 performs the parallel processing of the medical image obtained by imaging the endoscope 13a has been described. However, the present invention is not limited thereto, and parallel processing may be performed on each medical image 18 of the medical image group 17 acquired by another medical examination apparatus such as an ultrasonography apparatus or a radiation imaging apparatus.

In the above embodiments, hardware structures of processing units for executing various kinds of processing, such as the central control unit, the image acquisition unit 20, the part classifier 21, the classification result determination unit 22, the region-of-interest recognition unit 24, the recognition result acquisition unit 29, the image creation unit 30, the output controller 31, and the integrated determiner 60, are various processors shown below. The various processors include a central processing unit (CPU) that is a general-purpose processor that functions as various processing units by executing software (program), a programmable logic device (PLD) that is a processor whose circuit configuration can be changed after manufacture, such as field-programmable gate array (FPGA), a dedicated electrical circuit that is a processor having a circuit configuration designed exclusively for executing various types of processing, and the like.

One processing unit may be configured by one of various processors, or may be configured by a combination of two or more processors of the same or different kinds (for example, a combination of a plurality of FPGAs or a combination of a CPU and an FPGA). In addition, a plurality of processing units may be configured by one processor. As an example of configuring a plurality of processing units via one processor, first, as represented by a computer, such as a client or a server, there is a form in which one processor is configured by a combination of one or more CPUs and software, and this processor functions as a plurality of processing units. Second, as represented by a system-on-chip (SoC) or the like, there is a form of using a processor for realizing the function of the entire system including a plurality of processing units with one integrated circuit (IC) chip. Thus, various processing units are configured by using one or more of the above-described various processors as hardware structures.

More specifically, the hardware structure of these various processors is an electrical circuit (circuitry) in the form of a combination of circuit elements, such as semiconductor elements. The hardware structure of the storage unit is a storage device such as a hard disc drive (HDD) or a solid-state drive (SSD).

Further, from the above description, it is possible to understand the medical image processing apparatus described in Supplementary Notes 1 to 15 below and the endoscope system described in Supplementary Note 16.

### Supplementary Note 1

A medical image processing apparatus comprising:
a processor;
a classifier that classifies an imaged part in a medical image; and
a plurality of recognizers that recognize a region of interest in the medical image and correspond to each imaged part,
wherein the processor is configured to:
   acquire a plurality of medical images having a first medical image and a second medical image acquired at a later time point than the first medical image;
   acquire a classification result of the imaged part in the first medical image from the classifier;
   select the recognizer corresponding to the classification result of the first medical image from the plurality of recognizers; and
   acquire a recognition result of the region of interest in the second medical image from the selected recognizer.

### Supplementary Note 2

The medical image processing apparatus according to Supplementary Note 1,
wherein the processor is configured to perform
   classification of the imaged part in the second medical image by the classifier, and
   recognition of the region of interest in the second medical image by the recognizer in parallel processing.

### Supplementary Note 3

The medical image processing apparatus according to Supplementary Note 1 or 2,
wherein the processor is configured to provide a notification of the recognition result.

### Supplementary Note 4

The medical image processing apparatus according to Supplementary Note 3,
wherein the processor is configured to:
   create a display image using the recognition result; and
   provide the notification by displaying the display image on a screen.

### Supplementary Note 5

The medical image processing apparatus according to Supplementary Note 4,
wherein the processor is configured to perform
   classification of the imaged part in a third medical image acquired at a later time point than the second medical image by the classifier,
   recognition of the region of interest in the third medical image by the recognizer based on the imaged part in the second medical image, and
   creation of the display image of the recognition result in the second medical image in parallel processing.

### Supplementary Note 6

The medical image processing apparatus according to Supplementary Note 5,
wherein the processor is configured to create the display image by superimposing the recognition result on the third medical image.

### Supplementary Note 7

The medical image processing apparatus according to any one of Supplementary Notes 3 to 6,
wherein the processor is configured to provide a notification of a type of the recognizer used to recognize the region of interest in addition to the recognition result.

### Supplementary Note 8

The medical image processing apparatus according to any one of Supplementary Notes 1 to 7,
wherein the processor is configured to acquire the classification result using the first medical image and a plurality of medical images in a temporal vicinity of the first medical image acquired at an earlier time point than the second medical image.

### Supplementary Note 9

The medical image processing apparatus according to Supplementary Note 8,
wherein the processor is configured to select the recognizer according to the classification result and a selection history of the recognizer at a previous time point.

### Supplementary Note 10

The medical image processing apparatus according to Supplementary Note 9,
wherein the processor is configured to:
   weight the classification results of the plurality of medical images in the temporal vicinity based on the selection history; and
   select the recognizer using the weighted classification results.

### Supplementary Note 11

The medical image processing apparatus according to Supplementary Note 9,
wherein the processor is configured to:
   acquire, from the classifier, the classification result in which a degree of certainty is calculated for each imaged part; and
   continue to use the recognizer at the previous time point from the selection history in a case in which the degree of certainty is less than a specific reference value.

### Supplementary Note 12

The medical image processing apparatus according to any one of Supplementary Notes 1 to 11,
wherein each of the plurality of recognizers is a trained deep learning model trained on a different data set.

### Supplementary Note 13

The medical image processing apparatus according to Supplementary Note 12,
wherein the processor is configured to, in a case in which the classification result is a first part, select the recognizer trained on a data set including more images captured at the first part than images captured at a part different from the first part.

### Supplementary Note 14

The medical image processing apparatus according to any one of Supplementary Notes 1 to 13,
wherein each of the plurality of recognizers has a function of recognizing different regions of interest, and
the selected recognizer acquires the recognition result by executing at least one of position detection, type determination, or region measurement for the region of interest in the medical image.

### Supplementary Note 15

The medical image processing apparatus according to Supplementary Note 1, further comprising:
a discriminator in which the classifier and the plurality of recognizers are integrated, the discriminator calculating an intermediate feature amount in the medical image, classifying the imaged part from the intermediate feature amount, and recognizing the region of interest from the intermediate feature amount and the imaged part,
wherein the processor is configured to:
   acquire a first intermediate feature amount calculated from the first medical image and a second intermediate feature amount calculated from the second medical image;
   acquire the classification result based on the first intermediate feature amount;
   select a deep learning model corresponding to the classification result in the first intermediate feature amount from a plurality of deep learning models; and
   acquire the recognition result of the region of interest in the second intermediate feature amount from the selected deep learning model.

### Supplementary Note 16

An endoscope system comprising:
the medical image processing apparatus according to any one of Supplementary Notes 1 to 15; and
an endoscope that acquires the medical image.

### EXPLANATION OF REFERENCES

10: medical image processing system
11: medical image processing apparatus
12: database
13: endoscope system
13a: endoscope
14: user interface
17: medical image group
18: medical image
20: image acquisition unit
21: part classifier
22: classification result determination unit
24: region-of-interest recognition unit
25: classification result storage unit
26: first recognizer
27: second recognizer
28: third recognizer
29: recognition result acquisition unit
30: image creation unit
31: output controller
40: display image
41: marker
42: type display field
43: size display field
44: used recognizer display field
45: schema diagram
50: medical image processing apparatus
60: integrated determiner
61: intermediate feature amount calculation unit
62: part classification unit
63: classification result acquisition unit
64: first recognition unit
65: second recognition unit
66: third recognition unit
R: region of interest
ST100 to ST180: step
T0 to T5: time point

What is claimed is:

1. A medical image processing apparatus comprising:
one or more processors;
a classifier that classifies an imaged part in a medical image; and
a plurality of recognizers that recognize a region of interest in the medical image and correspond to each different imaged part,
wherein the one or more processors are configured to:
acquire a plurality of medical images having a first medical image and a second medical image acquired at a later time point than the first medical image;
acquire a classification result of the imaged part in the first medical image from the classifier;
select the recognizer corresponding to the classification result of the first medical image from the plurality of recognizers; and
acquire a recognition result of the region of interest in the second medical image from the selected recognizer.

2. The medical image processing apparatus according to claim 1,
wherein the one or more processors are configured to perform parallel processing of:
classification of the imaged part in the second medical image by the classifier; and
recognition of the region of interest in the second medical image by the recognizer.

3. The medical image processing apparatus according to claim 1,
wherein the one or more processors are configured to provide a notification of the recognition result.

4. The medical image processing apparatus according to claim 3,
wherein the one or more processors are configured to:
create a display image using the recognition result; and
provide the notification by displaying the display image on a screen.

5. The medical image processing apparatus according to claim 4,
wherein the one or more processors are configured to perform parallel processing of:
classification of the imaged part in a third medical image acquired at a later time point than the second medical image by the classifier;
recognition of the region of interest in the third medical image by the recognizer based on the imaged part in the second medical image; and
creation of the display image of the recognition result in the second medical image.

6. The medical image processing apparatus according to claim 5,
wherein the one or more processors are configured to create the display image by superimposing the recognition result on the third medical image.

7. The medical image processing apparatus according to claim 3,
wherein the one or more processors are configured to provide a notification of a type of the recognizer used to recognize the region of interest, in addition to the recognition result.

8. The medical image processing apparatus according to claim 1,
wherein the one or more processors are configured to acquire the classification result using the first medical image and a plurality of medical images in a temporal vicinity of the first medical image acquired at an earlier time point than the second medical image.

9. The medical image processing apparatus according to claim 8,
wherein the one or more processors are configured to select the recognizer according to the classification result and a selection history of the recognizer at a previous time point.

10. The medical image processing apparatus according to claim 9,
wherein the one or more processors are configured to:
weight the classification results of the plurality of medical images in the temporal vicinity based on the selection history; and
select the recognizer using the weighted classification results.

11. The medical image processing apparatus according to claim 9,
wherein the one or more processors are configured to:
acquire, from the classifier, the classification result in which a degree of certainty is calculated for each imaged part; and
continue to use the recognizer at the previous time point from the selection history, in a case in which the degree of certainty is less than a specific reference value.

12. The medical image processing apparatus according to claim 1,
wherein each of the plurality of recognizers is a trained deep learning model trained on a different data set.

13. The medical image processing apparatus according to claim 12,
wherein the one or more processors are configured to, in a case in which the classification result is a first part, select the recognizer trained on a data set including more images captured at the first part than images captured at a part different from the first part.

14. The medical image processing apparatus according to claim 1,
wherein each of the plurality of recognizers has a function of recognizing different regions of interest, and
the selected recognizer acquires the recognition result by executing at least one of position detection, type determination, or region measurement for the region of interest in the medical image.

15. The medical image processing apparatus according to claim 1, further comprising:
a discriminator in which the classifier and the plurality of recognizers are integrated, the discriminator calculating an intermediate feature amount in the medical image, classifying the imaged part from the intermediate feature amount, and recognizing the region of interest from the intermediate feature amount and the imaged part,
wherein the one or more processors are configured to:
acquire a first intermediate feature amount calculated from the first medical image and a second intermediate feature amount calculated from the second medical image;
acquire the classification result based on the first intermediate feature amount;
select a deep learning model corresponding to the classification result in the first intermediate feature amount from a plurality of deep learning models; and
acquire the recognition result of the region of interest in the second intermediate feature amount from the selected deep learning model.

16. An endoscope system comprising:
the medical image processing apparatus according to claim 1; and
an endoscope that acquires the medical image.
